(19) United States
(12) Patent Application Publication
(10) Pub. No.: US 2006/0084717 A1
(43) Pub. Date: Apr. 20, 2006

(54) Dental self-etching composition and method of use

(76) Inventors: Gordon S. Cohen, Madison, CT (US); Weitao Jia, Wallingford, CT (US)

Correspondence Address: Ann M. Knab;Pentron Corporation, 53 North Plains Industrial Road, Wallingford, CT, 06492, US

(21) Appl. No.: 11/255,121

(22) Filed: Oct. 20, 2005

Related U.S. Application Data
(60) Provisional application No. 60/620,646, filed on Oct. 20, 2004.

(51) Int. Cl. A61K 6/08

(52) U.S. Cl. 523/116

(57) ABSTRACT

A self-etching primer composition and method for etching and treating a tooth surface prior to restoration. The self-etching primer composition contains DOPA. In practice, the composition is applied to the tooth surface and not subsequently washed prior to application of an adhesive or other restorative material.

### CROSS-REFERENCE TO RELATED APPLICATIONS

This application claims priority to U.S. Provisional Application No. 60/620,646 filed Oct. 20, 2004, entitled Dental Self-Etching Composition And Method Of Use, which is incorporated herein by reference.

### TECHNICAL FIELD

The present invention is generally directed to compositions and methods for preparing the surfaces of teeth prior to their repair or restoration, including cavity fillings, core build-ups, restorative cementations, and root canal treatments, and in particular to a self-etching composition and method of use thereof.

### BRIEF DESCRIPTION OF THE RELATED ART

Methods and compositions for improving the adhesion of resins to hard tissue, i.e., dentin or enamel, is an ongoing goal in the dental arts. Improved adhesion leads to longer lasting restorations and reduced tooth sensitivity. Numerous methods for preparing teeth for the application of a dental restorative material (such as a sealant, filling material, cementation of indirect dental restorations or the like) have accordingly been developed, including acid etch and priming steps. Unfortunately, such steps have increased operating time and complexity.

Acid etchants are commonly thought to remove smear layers and demineralize the tooth surfaces so as to promote effective mechanical bonding of the restorative material. However, the use of an etchant has a disadvantage, in that it must be washed off after application, requiring the time-consuming procedure of application, washing and drying. A further disadvantage of etchants is the perception that use of strong etchants can increase dental sensitivity in some patients.

In addition to acid etch procedures, adhesive strength is also improved by use of a primer. Primers are generally surface-active compounds that exhibit both an affinity for dentin and adhesive resin systems and participate in the polymerization process, thereby promoting adhesion between the primarily hydrophilic dentin and the predominantly hydrophobic polymeric adhesives or monomers from which they are formed. Primers are applied to dentin in solution form, such commonly used solvents including acetone, ethanol, water, and various mixed solvent systems. U.S. Pat. Nos. 4,588,756 and 5789,610 to Bowen disclose the use of N-phenylglycine (NPG) and the adduct of N(p-tolyl)glycine and glycidyl methacrylate (“NTG-GMA”), as primers, which, in addition to their surface-active properties, also function as co-initiators or activators during interfacial polymerization. U.S. Pat. No. 6,458,869 to Antonucci teaches using N-phenyliminodiacetic acid (PIDAA) as a dentin bonding self-etching primer for dentin bonding. While effective for promoting bonding, primers are often applied using an additional step. Furthermore, amino acids, in general, are easily oxidized and in turn, the solutions of amino acids become a dark brown color in a matter of weeks, if not days.

There accordingly remains a need in the art for improved compositions which improve adhesion, which do not increase tooth sensitivity, and yet which can be applied in a fewer number of steps.

### SUMMARY OF THE INVENTION

The above-described drawbacks and disadvantages are alleviated by a self-etching primer composition comprising dihydeoxyphenylalanine (DOPA), which is a highly reactive amino acid. DOPA is present in quantities effective to provide etching and priming, generally in the range from about 0.1 to about 10 percent and more preferably from about 0.5 to about 5.0 weight percent of the total composition. In a particularly advantageous feature, the composition will increase the adhesiveness of the tooth structure without the need for washing the composition from the tooth surface. This composition can accordingly be provided as a single component material for ease of application and storage. The composition may further include a desensitizing agent and an antimicrobial agent, in the form of an aldehyde having from 2 to about 20 carbon atoms, preferably glutaraldehyde, in an amount effective to decrease dental sensitivity. Metallic ion salts such as potassium nitrate or calcium chloride can also be added. Fluoride or a fluoride source may also be added to the composition.

In accordance with the method of use, the above-described composition is physically contacted with the tooth structure, and then at least partially dried prior to application of an adhesive or other restorative composition. No intermediate washing step or second primer application step is required.

### DETAILED DESCRIPTION OF THE PREFERRED EMBODIMENTS

The inventors hereof have unexpectedly discovered that DOPA is effective to simultaneously etch and prime a tooth to receive a dental restoration. When DOPA is dissolved in a diluted mineral acid solution, such as HCl, the resulting self-etch primer is very stable in storage. Examples of preferred DOPA forms include Levodopa (L-DOPA), a naturally occurring form of DOPA, D-DOPA and DL-DOPA.

A self-etching, priming composition in accordance with the present invention accordingly comprises a solution of a DOPA in combination with a common diluted mineral acid such as HCl, HNO₃, H₃PO₄, H₂SO₄, and similar acids. It is preferable that the diluted acid has a concentration of the acid normality of about two or less.

The DOPA component is present in an amount of from about 0.1 to about 50 weight percent and more preferably from about 0.2 to about 10 weight percent and most preferably from about 0.5 to about 5.0 weight percent of the total composition. The acid component is present in an amount of from about 5 to about 99 weight percent and more preferably from about 40 to about 98 weight percent of the total composition.

This DOPA solution can be used along with other self-etching primers such as those disclosed in commonly owned U.S. Pat. No. 6,592,372 to Jia et al. to further enhance the bonding effect.

Additional components that may be included in the composition include monomers having both olefinic unsaturation and terminal —SO3 groups, such as an —SO3H group. Examples of such compounds include 2-acrylamido-2-methyl-propanesulfonic acid (AMPS) and its derivatives, 2-sulfoethyl methacrylate (SEM) and its derivatives, and 3-sulfopropyl methacrylate (SPM) and its derivatives. Examples of derivatives include sulfonic acid salts of AMPS, SEM and SPM, and hydrolytically active esters of AMPS, SEM, and SPM. AMPS compounds are available from Lubrizol Corporation, Wickliffe, Ohio. SEM and SPM compounds are available from Polyscience, Inc., PA.

Suitable salt countering include without limitation alkali and alkaline earth metals. Suitable ester moieties include without limitation lower alkyl groups, for example methyl, ethyl, propyl, isopropyl, and the like, as well as aromatic groups such as benzyl. The —SO₃ terminated monomers are present in the solution in amounts from about 0.1 to about 50 weight percent, more preferably about 0.5 to about 20 weight percent, and most preferably from about 1 to about 10 weight percent of the total composition.

The self-etching/primer composition may further include an aldehyde in an amount effective to decrease sensitivity at the site of the dental restoration. Suitable aldehydes include aliphatic aldehydes having from 2 to about 20, preferably from 2 to about 10, and most preferably from 2 to about 6 carbon atoms. Aromatic and heteroaromatic aldehydes having from 6 to about 20 carbons may also be used. Dialdehydes are also within the scope of the invention. Exemplary aldehydes include but are not limited to acetic aldehyde, propionaldehyde, glyoxal, benzaldehyde, vanilline, salicylic aldehyde, o-phthalic aldehyde, anisaldehyde, furfural, and the like. Preferably, the desensitizing agent is glutaraldehyde.

When present, effective quantities of aldehyde are readily determined by one of ordinary skill in the art. In general, effective quantities comprise from about 0.1 to about 20% by weight, preferably from about 0.2 to about 10% by weight, and most preferably from about 0.5 to about 3% by weight of the total composition.

The self-etching/primer adhesive composition may further include an optional fluoride source. Suitable fluoride sources which are compatible with the components of the composition include, for example, sodium fluoride, stannous fluoride, sodium monofluorophosphate, calcium fluoride, calcium fluorophosphate, and the like. When present, fluoride-releasing compounds are used in quantifies of up to about 2% by weight of the total composition.

The self-etching/primer adhesive composition may further comprise additional, optional components for enhancing the priming, bonding, cleaning or conditioning effect of the composition. Such components include chemicals containing polymerizable double bonds such as those of methacrylic acid, ester, or similar groups; additional acids with good or limited solubility in water; surfactants; and dyes such as methylene blue, medicants, such as chlorohexadine and its derivatives, pH indicators, and the like. More specifically, examples of useful priming components include 2-hydroxyethylmethacrylate, glyceryl methacrylate, hydroxypropylmethacrylates, itaconic acid, ethyleneglycolmethacrylate, maleic acid 2-(methacryloyloxy)ethyl phosphate, trimethylolpropane trimethacrylate (TMPTMA) and other polymerizable (meth)acrylic monomers/oligomers known in the field. Examples of light curing initiators include 2,4,6-trimethylbenzoyl diphenyl phosphine oxide (Lucirin TPO available from BASF, Germany) and the like. These optional components are generally present in amounts in the range of up to about 50 weight percent.

The inclusion of a pH indicator can indicate a change in pH of the self-etch composition due to the neutralization effect of the etching process from the tooth minerals. Examples of suitable pH indicators include methyl red, which turns red in acid; yellow when the pH approaches neutral; and litmus, which turns red in acid and blue when the pH becomes slightly basic. The pH indicator also enables the practitioner to see where the solution is being applied.

The inclusion of a dye such as methylene blue in small quantities of less than 0.1 percent can provides a blue tint to the composition when it is applied to the tooth surface. In practice, the primer containing the dye is applied to the tooth surface. After a short period of waiting and drying of the primer, a layer of a dental adhesive is then applied onto the primed surface and is subjected to photocuring for a time period from about 5 to about 60 seconds, after which time the blue color on the treated tooth surface diminishes and the adhesive layer appears colorless.

The etchant/primer compositions further include a solvent. Such a solvent system includes water and/or a polar solvent that is partially or totally miscible with water. For dental applications, a suitable solvent system is one that completely wets and diffuses into the conditioned surface of enamel, and particularly dentin, in a clinically acceptable period of time (on the order of about 15 to about 180 seconds). Preferred organic solvents include low molecular weight ketones, such as acetone and methyl ethyl ketone, which are readily soluble in water over a wide concentration range, or a low molecular weight alcohol, such as ethanol or propanol. Other solvents include polar aprotic liquids such as dimethylformamide, dimethylacetamide and dimethylsulfoxide. Water, ethanol, acetone, or a mixed solvent system of water and acetone are preferred. In such a solvent system, the amount, by volume, of acetone may range from about 5 to about 50% acetone, with the remainder being water.

The solvent serves the purpose of assuring that the self-etching/primer compound contacts all exposed dentin surfaces so that the self-etching/primer compound can function successfully. Thus, the solvent system must appropriately reduce the viscosity of the etchant/primer compound as well as provide a suitable surface tension such that the composition may penetrate the smallest cracks, fissures or pores in the dentin surface to assure suitable contact of the polymerized adhesive component with the dentin. In some instances, to provide the appropriate surface tension, a surfactant may be employed. In most instances, as when the self-etching/primer compound is combined with an adhesive monomer system, it is preferred that the solvent system used to dissolve the self-etching/primer compound also be miscible with the solvent system employed to dissolve the adhesive monomer system and/or be capable of dissolving the adhesive monomer system itself.

The amount of solvent used is 100 weight percent less the total amount of other components, preferably 30 to 99 weight percent, more preferably 40 to 99 weight percent of the total composition. Distilled or deionized water is preferred, as it does not contain impurities potentially harmful to the adhesive properties of the solution. When volatile solvents such as ethanol or acetone are used in the composition, the amount of water may be decreased to as low as 2 percent.

The self-etching/primer adhesive composition may be applied directly to the prepared tooth surface. The composition may be dispensed from a conventional push syringe, squeeze bottle, elongated plastic tubular tip, a metallic cannula, a single dose package or applied with a brush. After a specified time ranging from 5 to 120 seconds, preferably 10 to 60 seconds, the tooth surface is lightly dried. Washing is not required. After applying the tooth surface treatment composition, a polymerizable dental adhesive system may be applied, dried, and optionally cured, followed by application and curing of a dental restorative material. The dental adhesive bonds to the tooth without the need for the tooth to be washed. Alternatively, the dental adhesive system may be included in the self-etching/primer adhesive composition, combining the application of the self-etching/primer and the application of the dental adhesive into a single step.

Suitable dental adhesives and restoratives are those conventional in the art. The term ‘dental adhesive’ and the like as used herein can apply to a wide range of materials that can effect a bond to both conditioned enamel and dentin. At a minimum, the dental adhesive contains a polymerizable resin component or components necessary to effect the initiation and acceleration of polymerization by visible or actinic light or by chemical means, and a polymerizable monomer or monomers containing anionic functionality such as a phosphate or carboxylic (COOH) acid function. Examples of monomers include triethylene glycol dimethacrylate (hereinafter TEGDMA), 2-hydroxyethylmethacrylate (hereinafter HEMA), 2,2-bis[p-(2′-hydroxy-3′-methacryloxypropoxy)phenyl]propane (hereinafter Bis-GMA), polyurethane dimethacrylates (hereinafter PUDMA), trimethylolpropane trimethacrylate (hereinafter TMPTMA), and the like. This dental adhesive may be in the form of a self-priming adhesive that further contains a volatile solvent such as acetone, ethanol, and mixtures thereof. Water may also be used as a solvent. The dental adhesive may comprise a one-component material, or may alternatively have two components. The second component of the dental adhesive may contain initiators and/or accelerators, to facilitate chemical curing alone or combined with curing upon exposure to actinic light to provide a dual-cure mode of polymerization. Examples of substances that facilitate self-curing of dental adhesives include for example, BPO, DHEPT, and aromatic sulfinic acid salts. One useful dental adhesive includes Bond-1® and Bond-It® (both available from Pentron Clinical Technologies, LLC). Preferred dental adhesives are cured by exposure to light, preferably visible light.

Useful dental restorative materials or cements include amalgam and non-amalgam dental restoratives. Examples of useful non-amalgam materials include compomer restorative, composite resin restorative, glass ionomer-resin restorative, glass ionomer-resin luting cement, resin cement and resin dental sealant.

The composition, when applied to a tooth, enhances the adhesiveness of the tooth without the need for washing or a second application step. The multi-step bonding protocols typical of current commercial adhesive systems generally tend to be a source of material waste and unreasonable technique sensitivity. The present self-etching/primer adhesive compositions not only reduce the number of steps normally involved in preparing a substrate surface and applying the adhesive monomer system (from 3 or 4 steps to 1 or 2 steps), but less waste and improved restorative or sealant results are obtained.

Furthermore, although conventional aggressive etchants are effective in cleaning the surface of dentin for improved wetting by diffusion of the components of the adhesive system, they can also weaken the underlying sound dentin by excessive demineralization and disruption of collagen fibrils. These types of etchants typically require an aqueous rinse step to remove residual acid and soluble by-products. Also, the depth of demineralized, altered dentin resulting from the use of aggressive etchants may exceed the depth to which an adhesive resin can penetrate the dentin, resulting in a weakened, partially reinforced hybrid dentin zone, and thereby become vulnerable to failure. In contrast, the present composition is milder and may be used as single step etchant and primer compositions without subsequent rinsing since they are also effective in the presence of water and/or aqueous solvents. Accordingly, while an aqueous rinse step, such as the type used with multi-step systems to remove residual acid and soluble by-products, may be used, it is unnecessary to employ such a rinse step.

Another advantage of the mildness of the present compositions is that sensitivity for the patient at the site of the restoration is reduced. Such sensitivity is reduced even further where an effective aldehyde (such as glutaraldehyde) is used.

The following Table 1 sets forth self-etch primer compositions of the invention.

TABLE 1

| Component | A | B | C | D |
|---|---|---|---|---|
| DOPA (wt. %) | 5 | 2 | 2 | 1 |
| 0.5 N HCL (wt. %) | 95 | 98 | 94 | 49 |
| Glutaraldehyde (25% concentration) (wt. %) | | | 4 | |
| AMPS (wt. %) | | | | 2.5 |
| Hydroxy ethyl methacrylate (wt. %) | | | | 46 |
| TMPTMA (wt. %) | | | | 1 |
| Lucirin TPO (wt. %) | | | | 0.5 |

The following non-limiting examples illustrate the invention.

### EXAMPLES

Etchant solutions of the invention comprising the compositions set forth in Table 1 were used to test the bond strength of adhesives used in conjunction with the self-etch primer compositions of the invention. Tooth samples were prepared by mounting each tooth with a cold-cured acrylic in a cylinder form leaving the crown portion exposed. Each test group consisted of 5 tooth samples. Occlusal dentin was then exposed by cutting off the enamel portion of the tooth crown using a slow speed diamond wheel saw, Model 650 (South Bay Technologies, Inc.). The exposed dentin was then subjected to SiC abrasive paper through 600 grits. After the dentin surface was cleaned and water rinsed, it was lightly dried with a jet of air for 2-3 seconds to remove apparent water on the surface. Then the experimental self-etching primer and/or adhesive or the All-In-One adhesive were applied onto the tooth surface with a disposable brush tip.

For self-etch primers, a coat of the primer was brushed onto the tooth surface and left there for about 30 seconds and then air blown briefly for 2 seconds to dry or alternatively, blotted dry with a Kimwipes tissue or brushed off with a dry brush tip. For the samples using 37% H3PO4 as an etchant, the etching gel was applied onto the tooth surface, left for 20 seconds, and then flushed with water for at least 10 seconds to remove the acid, followed by blotting dry with a Kimwipes tissue.

Next a coat of a resin adhesive was applied onto the primed tooth surface and air dried for about 10 seconds to remove the solvent within the adhesive. The adhesive surface was cured for 10 seconds with visible light using an Optilux 400 light curing unit (available from Demetron/Kerr) at the radiation intensity of about 600 mw/cm2. The adhesive remaining on the brush from the first application of adhesive was brushed onto the cured adhesive surface.

An Ultradent dentin bonding device/jig (available from Ultradent, UT) was used for mounting the tooth bonding sample and making a composite cylinder on top of the cured adhesive surface. A Simile® A2 shade composite (available from Pentron Clinical Technologies, LLC) was used for the composite button build-up with a diameter of 2.38 mm and a thickness of about 2 mm. The composite was then light cured for 40 seconds from the top only.

The Ultradent device was removed and the bonded tooth sample was left in water at a temperature of 37° C. for 24 hours before testing. The bonding test was done in push-shear mold using the Ultradent device in conjunction with an ATS device under a crosshead speed of 0.02 in/min. The load at which the composite button was broken/fractured from the tooth was recorded and the bonding strength was calculated using the maximum load divided by the composite cylinder's surface area and expressed in megapascals (MPa). The standard deviation was then also calculated based on each group of testing samples. The following Table 2 provides the bond strength results.

TABLE 2

| | | Dentin | Enamel |
|---|---|---|---|
| | Adhesives (lot#) | Bonding | Bonding |
| Experimental Self- | used in conjunction | Strength | Strength |
| Etch Compositions | with the primer | (MPa, SD) | (MPa, SD) |
| A | Bond 1 ®* (#97806) | 24.8 (3.4) | 22.9 (4.6) |
| B | Bond 1 ®* (#97806) | 27.6 (6.9) | 26.3 (9.1) |
| B | NanoBond ®* (#69813) | 23.6 (4.4) | |
| C | Bond 1 ®* (#10073) | 32.5 (3.0) | 27.3 (6.5) |
| D | Bond-1 ®* (#10073) | 32.0 (2.6) | 30.1 (3.4) |
| D | Clearfil SE BOND** | 24.2 (3.7) | |
| | Bond Resin (00400A) | | |
| D | Bond-It ® VLC* | 28.2 (5.5) | 35.2 (3.4) |
| 37% H3PO4 etchant | Bond 1 ®* (#97806) | 27.5 (6.0) | 23.6 (3.2) |
| NanoBond ®* Self- | NanoBond ®* adhesive | 21.6 (3.2) | 21.8 (2.3) |
| Etch Primer (#69813) | | | |

*Bond 1, Bond-It and NanoBond dental adhesive products are from Pentron Clinical Technologies, LLC.
**Clearfil SE Bond dental adhesive product is from Kuraray Co., Ltd Corporation, Japan.

As shown in Table 2 the self-etching compositions of the invention show the same or better bonding results, but using less steps in the application of the etchant to the tooth surface.

It should be noted that Bond-It® VLC adhesive is a solvent-free resin adhesive in comparison to the other adhesives used in the examples in Table 2 above, which adhesives contain a solvent and require further air drying upon application. The use of the solvent-free adhesive in combination with the self-etch primer composition described herein provides a facile process and further reduces the presence of trapped air in the adhesive layer.

The foregoing description illustrates preferred embodiments of the invention. However, concepts employed may, based upon such description, be employed in other embodiments without departing from the scope of the invention. Accordingly, the following claims are intended to recite the invention broadly, as well as in the specific forms herein.

What is claimed is:

1. A self-etching primer composition comprising:
about 0.1 to about 50 weight percent of dihydroxyphenylalanine (DOPA).

2. The self-etching primer of claim 1 further comprising a mineral acid solvent in an amount of from about 40 to about 99 weight percent.

3. The self-etching primer of claim 2 wherein the mineral acid solvent is a diluted acid.

4. The self-etching primer of claim 2 wherein the mineral solvent comprises an inorganic acid selected from the group consisting of HCl, HNO₃, H₃PO₄, H₂SO₄, and a mixture thereof.

5. The self-etching primer of claim 1 further comprising a compound comprising about 0.1 to about 50 weight percent of an olefinically unsaturated, —SO₃ terminated monomer selected from the group consisting of AMPS, AMPS derivatives, SEM, SEM derivatives, SPM, SPM derivatives, or a mixture comprising at least one of the foregoing monomers.

6. The self-etching primer of claim 5 wherein the SO₃ terminated monomer further comprises a salt of an alkali or alkaline earth metal.

7. The self-etching primer of claim 1 wherein DOPA comprises Levodopa (L-DOPA), a naturally occurring form of DOPA, D-DOPA DL-DOPA or a mixture thereof.

8. The self-etching primer of claim 1 further comprising an aldehyde in an amount of about 0.1 to about 20 weight percent.

9. The self-etching primer of claim 8 wherein the aldehyde comprises acetic aldehyde, propionaldehyde, glyoxal, benzaldehyde, vanilline, salicylic aldehyde, o-phthalic aldehyde, anisaldehyde, furfural, or glutaraldehyde.

10. The self-etching primer of claim 1 further comprising from 0 to about 2 weight percent of a fluoride component.

11. The self-etching primer of claim 10 wherein the fluoride comprises sodium fluoride, stannous fluoride, sodium monofluorophosphate, calcium fluoride, or calcium fluorophosphates.

12. The self-etching primer of claim 1 further comprising a polymerizable resin in an amount of up to about 50 weight percent.

13. The self-etching primer of claim 12 wherein the polymerizable resin comprises one or more of a (meth)acrylic monomer or oligomer.

14. The self-etching primer of claim 13 wherein the (meth)acrylic monomer or oligomer comprises one or more of 2-hydroxyethylmethacrylate, glyceryl methacrylate, hydroxypropylmethacrylates, itaconic acid, ethyleneglycolmethacrylate, maleic acid 2-(methacryloyloxy)ethyl phosphate, and trimethylolpropane trimethacrylate.

15. The self-etching primer of claim 1 further comprising an additive selected from the group consisting of a surfactant, dye, pH indicator, medicant, light cure initiators, and mixtures thereof.

16. The self-etching primer of claim 15 wherein the light cure initiator comprises 2,4,6-trimethylbenzoyl diphenyl phosphine oxide.

17. The self-etching primer of claim 15 wherein the pH indicator comprises methyl red or litmus.

18. The self-etching primer of claim 15 wherein the medicant comprises chlorohexadine or its derivatives.

19. The self-etching primer of claim 15 wherein the dye comprises methylene blue.

20. The self-etching primer of claim 2 further comprising a second solvent in an amount of about 30 to about 99 weight percent of the total composition.

21. The self-etching primer of claim 20 wherein the second solvent comprises a low molecular weight ketone, a low molecular weight alcohol, a polar aprotic liquid, water or a mixture thereof.

22. The self-etching primer of claim 21 wherein the low molecular weight ketone comprises acetone, methyl ethyl ketone, or a mixture thereof.

23. The self-etching primer of claim 21 wherein the low molecular weight alcohol comprises ethanol, propanol or a mixture thereof.

24. The self-etching primer of claim 21 wherein the polar aprotic liquid comprises dimethylformamide, dimethylacetamide and dimethylsulfoxide or a mixture thereof.

25. The self-etching primer of claim 20 wherein the second solvent comprises water, ethanol and acetone.

26. The self-etching primer of claim 20 wherein the second solvent comprises water and acetone.

27. The self-etching primer of claim 2 further comprising a dental adhesive composition.

28. The self-etching primer of claim 27 wherein the dental adhesive composition comprises a polymerizable resin component.

29. The self-etching primer of claim 28 wherein the polymerizable resin component comprises triethylene glycol dimethacrylate (hereinafter TEGDMA), 2-hydroxyethylmethacrylate (hereinafter HEMA), 2,2-bis[p-(2′-hydroxy-3′-methacryloxypropoxy)phenyl]propane (hereinafter Bis-GMA), polyurethane dimethacrylates (hereinafter PUDMA), trimethylolpropane trimethacrylate (hereinafter TMPTMA), or mixtures thereof.

30. The self-etching primer of claim 27 wherein the dental adhesive composition further comprises an initiator, accelerator, or mixture thereof.

31. The self-etching primer of claim 27 wherein the dental adhesive is solvent-free.

32. The self-etching primer of claim 27 wherein the dental adhesive comprises a solvent.

33. The self-etching primer of claim 32 wherein the solvent comprises a volatile solvent.

34. The self-etching primer of claim 33 wherein the volatile solvent comprises acetone, ethanol or a mixture thereof.

35. The self-etching primer of claim 27 wherein the dental adhesive is a two-component system.

36. A method of etching and treating a tooth to increase adhesiveness, comprising:
applying a self-etching primer composition comprising about 0.1 to about 50 weight percent of DOPA; and
applying an adhesive composition to the surface without applying a separate primer composition.

37. The method of etching and treating a tooth to increase adhesiveness as set forth in claim 36 wherein the self-etching primer composition further comprises one or more of 2-hydroxyethylmethacrylate, glyceryl methacrylate, hydroxypropylmethacrylates, itaconic acid, ethyleneglycolmethacrylate, maleic acid 2-(methacryloyloxy)ethyl phosphate, trimethylolpropane trimethacrylate, and 2,4,6-trimethylbenzoyl diphenyl phosphine oxide.

38. The method of etching and treating a tooth to increase adhesiveness as set forth in claim 36 wherein the self-etching primer composition further comprises a mineral acid solvent in an amount of from about 40 to about 99 weight percent.

39. The method of etching and treating a tooth to increase adhesiveness as set forth in claim 38 wherein the mineral acid solvent is a diluted acid.

40. The self-etching primer of claim 38 wherein the mineral solvent comprises an inorganic acid selected from the group consisting of HCl, HNO₃, H₃PO₄, H₂SO₄, and a mixture thereof.

41. The method of etching and treating a tooth to increase adhesiveness as set forth in claim 36 wherein the self-etching primer composition further comprises a compound comprising about 0.1 to about 50 weight percent of an olefinically unsaturated, —SO₃ terminated monomer selected from the group consisting of AMPS, AMPS derivatives, SEM, SEM derivatives, SPM, SPM derivatives, or a mixture comprising at least one of the foregoing monomers.

42. The method of etching and treating a tooth to increase adhesiveness as set forth in claim 41 wherein the olefinically unsaturated, —SO₃ terminated monomer further comprises a salt of an alkali or alkaline earth metal.

43. The method of etching and treating a tooth to increase adhesiveness as set forth in claim 36 wherein the self-etching primer composition further comprises an aldehyde in an amount of about 0.1 to about 20 weight percent.

44. The method of etching and treating a tooth to increase adhesiveness as set forth in claim 43 wherein the aldehyde is glutaraldehyde.

45. The method of etching and treating a tooth to increase adhesiveness as set forth in claim 36 wherein the self-etching primer composition further comprises from 0 to about 2 percent by weight of a fluoride component.

46. The method of etching and treating a tooth to increase adhesiveness as set forth in claim 45 wherein the fluoride comprises sodium fluoride, stannous fluoride, sodium monofluorophosphate, or calcium fluorophosphates.

47. The method of etching and treating a tooth to increase adhesiveness as set forth in claim 36 wherein the self-etching primer composition further comprises an polymerizable resin in an amount of up to about 50 weight percent.

48. The method of etching and treating a tooth to increase adhesiveness as set forth in claim 47 wherein the polymerizable resin comprises one or more of 2-hydroxyethylmethacrylate, glyceryl methacrylate, hydroxypropylmethacrylates, itaconic acid, ethyleneglycolmethacrylate, maleic acid 2-(methacryloyloxy)ethyl phosphate, and trimethylolpropane trimethacrylate.

49. The method of etching and treating a tooth to increase adhesiveness as set forth in claim 36 further comprising an additive selected from the group consisting of a surfactant, dye, pH indicator, medicant, light cure initiators, or mixtures thereof.

50. The method of etching and treating a tooth to increase adhesiveness as set forth in claim 49 wherein the light cure initiator comprises 2,4,6-trimethylbenzoyl diphenyl phosphine oxide.

51. The method of etching and treating a tooth to increase adhesiveness as set forth in claim 49 wherein the pH indicator comprises methyl red.

52. The method of etching and treating a tooth to increase adhesiveness as set forth in claim 49 wherein the medicant comprises chlorohexadine or its derivatives.

53. The method of etching and treating a tooth to increase adhesiveness as set forth in claim 49 wherein the dye comprises methylene blue.

54. The method of etching and treating a tooth to increase adhesiveness as set forth in claim 36 wherein the self-etching primer further comprises a second solvent in an amount of about 30 to about 99 weight percent of the total composition.

55. The method of etching and treating a tooth to increase adhesiveness as set forth in claim 54 wherein the second solvent comprises a low molecular weight ketone, a low molecular weight alcohol, a polar aprotic liquid, water, or a mixture thereof.

56. The method of etching and treating a tooth to increase adhesiveness as set forth in claim 55 wherein the low molecular weight ketone comprises acetone, methyl ethyl ketone, or a mixture thereof.

57. The method of etching and treating a tooth to increase adhesiveness as set forth in claim 55 wherein the low molecular weight alcohol comprises ethanol, propanol or a mixture thereof.

58. The method of etching and treating a tooth to increase adhesiveness as set forth in claim 55 wherein the polar aprotic liquid comprises dimethylformamide, dimethylacetamide and dimethylsulfoxide or a mixture thereof.

59. The method of etching and treating a tooth to increase adhesiveness as set forth in claim 54 wherein the second solvent comprises water, ethanol and acetone.

60. The method of etching and treating a tooth to increase adhesiveness as set forth in claim 54 wherein the second solvent comprises water and acetone.

61. The method of etching and treating a tooth to increase adhesiveness as set forth in claim 36 wherein the dental adhesive composition comprises a polymerizable resin component.

62. The method of etching and treating a tooth to increase adhesiveness as set forth in claim 61 wherein the polymerizable resin component comprises triethylene glycol dimethacrylate (hereinafter TEGDMA), 2-hydroxyethylmethacrylate (hereinafter HEMA), 2,2-bis[p-(2′-hydroxy-3′-methacryloxypropoxy)phenyl]propane (hereinafter Bis-GMA), polyurethane dimethacrylates (hereinafter PUDMA), trimethylolpropane trimethacrylate (hereinafter TMPTMA), or mixtures thereof.

63. The method of etching and treating a tooth to increase adhesiveness as set forth in claim 61 wherein the dental adhesive composition further comprises an initiator, accelerator, or mixture thereof.

64. The method of etching and treating a tooth to increase adhesiveness as set forth in claim 36 wherein the dental adhesive composition is solvent-free.

65. The method of etching and treating a tooth to increase adhesiveness as set forth in claim 36 wherein the dental adhesive comprises a solvent.

66. The method of etching and treating a tooth to increase adhesiveness as set forth in claim 65 wherein the solvent comprises a volatile solvent.

67. The method of etching and treating a tooth to increase adhesiveness as set forth in claim 65 wherein the volatile solvent comprises acetone, ethanol or a mixture thereof.

68. The method of etching and treating a tooth to increase adhesiveness as set forth in claim 36 wherein the adhesive composition is a two-component system.

69. The method of claim 36 wherein the self-etching primer composition is applied to the tooth by using a conventional push syringe, squeeze bottle, elongated plastic tubular tip, a metallic cannula, a single dose package or a brush.
